(19) United States
(12) Patent Application Publication
(10) Pub. No.: US 2006/0089542 A1
(43) Pub. Date: Apr. 27, 2006

(54) Mobile patient monitoring system with automatic data alerts

(75) Inventor: Alan Sands, Phoenix, AZ (US)

Correspondence Address: ETHERTON LAW GROUP, LLC, 5555 E. VAN BUREN STREET, SUITE 100, PHOENIX, AZ, 85008, US

(73) Assignee: Safe and Sound Solutions, Inc.

(21) Appl. No.: 11/149,416

(22) Filed: Jun. 8, 2005

Related U.S. Application Data
(60) Provisional application No. 60/622,337, filed on Oct. 25, 2004.

(51) Int. Cl. A61B 5/00; A61B 5/08; A61B 5/02; A61B 10/00

(52) U.S. Cl. 600/300; 600/565; 600/500; 600/529

(57) ABSTRACT

A system to increase compliance with patient monitoring protocols for patients with chronic disease. The system uses a wireless telecommunication device as the hub of the system. The hub is configured to increase patient compliance with a monitoring protocol by being integrated with a mobile device, such as a cellular phone or PDA, that the patient normally carries or wears. The hub is further configured to increase compliance by displaying games that incorporate monitored conditions and providing rewards to the patient when he complies with the monitoring protocol. The hub receives physiological data about the patient from a medical sensor then collates the sensed data with certain data input by the patient. The reading is transmitted to a server that uses a software application to automatically examine and interpret the data. Alerts are sent to the health care provider only when the reading is outside specified parameters. The health care provider may contact the patient about the outlying event via the network.

### CROSS-REFERENCE TO RELATED APPLICATIONS

This application claims the benefit of co-pending U.S. Provisional Application No. 60/622337 filed Oct. 25, 2004.

### FIELD OF INVENTION

This invention relates to the diagnosis of a plurality of physiologic functions. This invention relates particularly to mobile system for remotely monitoring and communicating physiological readings of a patient with chronic disease.

### BACKGROUND

Systems are known in the prior art that remotely measure physiological patient data and transmit it to a doctor's office so that the doctor can review the data and respond to the patient without having the patient visit the office. One known system comprises a sensor connected to a computer which, in turn, is networked via the internet to the doctor's computer. See, for example, U.S. Pat. No. 5,997,476 issued to Brown. These systems are known as patient-monitoring systems. These remote monitoring systems are particularly useful for patients with chronic diseases such as diabetes or asthma who must take certain readings numerous times a day to monitor the disease and prevent it from getting out of control.

The main problem with known patient-monitoring systems is that, for various reasons, patients simply don't use them and therefore become non-compliant with the monitoring protocol. Non-compliance defeats the goal improving the patient's health because minor deviations from a desired profile are left untreated until they become major deviations and cause acute health problems. Treatment for acute health problems generally requires more highly-trained health care providers and more urgent response than for non-acute problems, resulting in higher health costs. It is desirable to detect and treat minor deviations before they become acute so that the patients can be treated by the most cost-efficient level of health care providers in a non-exigent timeframe, thus reducing costs. That is, it is desirable to avoid acute problems and the associated cost of treatment by a specialized physician in an emergency situation. Chronic diseases can be controlled if monitored closely, thus enabling a diseased patient to live a relatively healthy life at a relatively low cost. Therefore, compliance with a monitoring protocol is highly desired.

Several fundamental factors contribute to noncompliance. First, the known systems are not mobile because they are physically connected by cable to a computer system or to a telephone or network connection to transmit the physiological data to a central location. This means that the patient must be at home or other designated physical location where the connection resides each time he transmits data. This is inconvenient for all patients and simply impractical for many active patients, which causes the patients to not comply with the monitoring protocol. Fortunately, wireless communications devices, such as cellular phones and email devices, have become wildly popular. It would be desirable to combine a monitoring system with mobile technology so that patients can conveniently comply with the monitoring protocol.

Another problem with the systems known in the prior art is that the data is transmitted directly to the doctor and the doctor must examine and interpret the data for each patient. This is a time- and labor-intensive process which is all the more frustrating because noncompliance can trigger false alarms of a patient's status; it is inefficient and expensive. It would be desirable to automatically examine and interpret the data so that the doctor has to be involved only when the patient is genuinely non-compliant with the treatment protocol or when the patient's data is outside preset parameters, indicating a health issue that needs to be addressed promptly.

Another problem with the known systems is that there is no incentive to comply with the monitoring protocol. While better health would seem to be a sufficient motivating factor, human nature being what it is, patients have demonstrated time and time again that they need additional incentive to take the actions and make the repeated measurements necessary for better health. It is known that people do what they enjoy, and that people enjoy games, visual and aural entertainment, and rewards. It is desirable to use such incentives motivate patients to comply with the monitoring protocol.

Therefore, an object of this invention is to provide a method and apparatus to increase compliance with patient monitoring protocols for patients with chronic disease. It is a further object to achieve this with a mobile system. It is a further object to provide incentive for a patient to take the necessary physiological measurements and otherwise comply with the monitoring protocol. It is another object to automatically examine and interpret the patient's data and to alert health care providers appropriately. It is another object to enable health care providers to provide information to patients promptly, especially in response to data deviations from the desired norm.

### SUMMARY OF THE INVENTION

The present invention is a system to increase compliance with patient monitoring protocols for patients with chronic disease. The present invention captures patient health information from sensors, relays this information wirelessly to a telecommunication device which, in turn, relays the information to a central repository. The data is stored and automatically analyzed and interpreted for those interested in the patients' well-being, such as health care providers, disease maintenance organizations or insurance companies. Feedback is provided to the patients to reduce health care costs and improve quality of life.

The system uses a telecommunication device as the hub of the system. The hub wirelessly receives physiological data about the patient from a medical sensor and transmits it to a server where the patient's record is updated with the current physiological data. In the preferred embodiment, the update triggers the server to query the patient to obtain additional data concurrent with the physiological data. In response to queries, the patient inputs patient data into the hub. The hub transmits it to the server where the patient record is updated again so that the physiological data and patient data are collated into a reading. The server uses a software application to automatically examine and interpret the reading. If the reading deviates from preset parameters, an alert is sent to a health care industry member affiliated with the patient who, in turn, transmits medical or other information to the patient via the server to the hub.

The hub is configured to increase patient compliance with a monitoring protocol by being integrated with a mobile device, such as a cellular phone or PDA, that the patient normally carries or wears. The hub is further configured to increase compliance by displaying games that incorporate monitored conditions and providing rewards to the patient when he complies with the monitoring protocol.

### BRIEF DESCRIPTION OF THE DRAWINGS

FIG. 1 is a schematic diagram of the technology of the present system.

FIG. 2 illustrates the relationship of the patient, care providers, and system.

FIG. 3 is a flow diagram illustrating a preferred method of use of the present system.

### DETAILED DESCRIPTION OF THE INVENTION

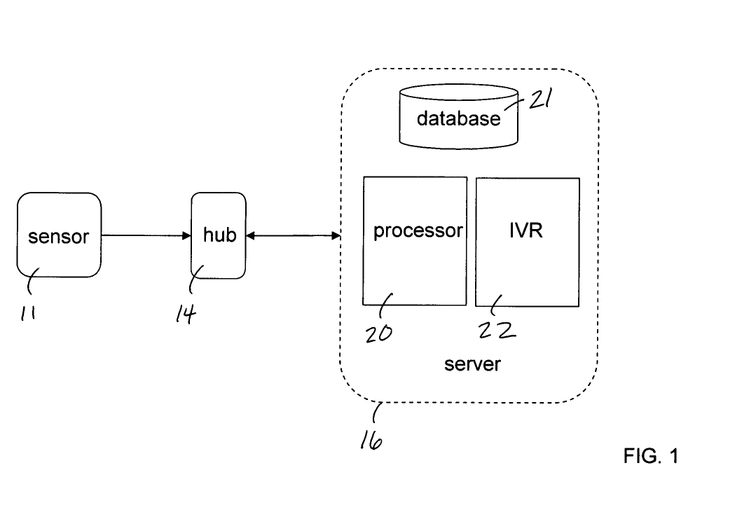

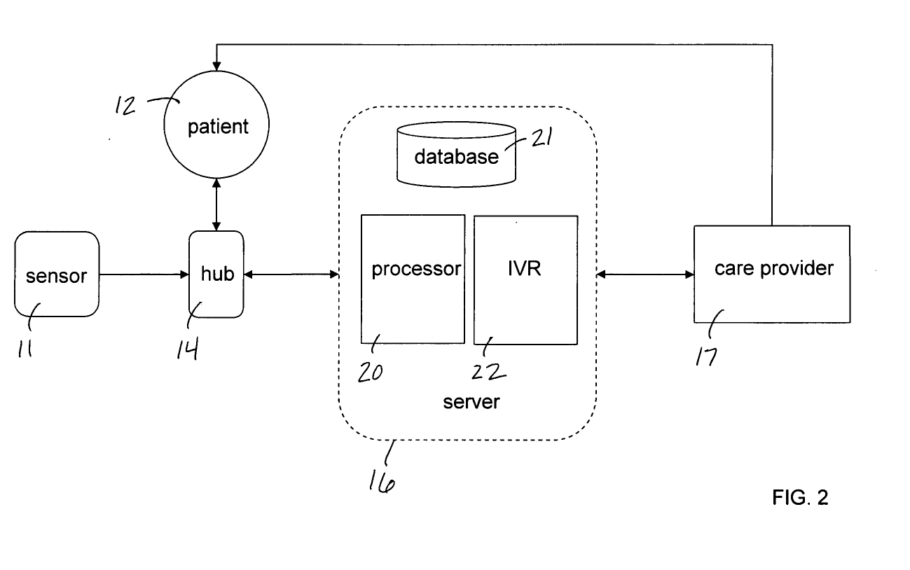

The technology of the present system is illustrated in FIG. 1. The relationship of the technology to the patient and care providers is shown in FIG. 2.

Sensor

A sensor 11 is used to measure at least one physiological measurement of a patient 12. Preferably, each sensor 11 supports internal storage of the measured values, along with a date and time stamp for each measurement. Additionally, the sensor 11 may store and transmit an identifier unique to the sensor 11 or to the patient 12. The physiological measurement, alone or in combination with other data transmitted by the sensor 11, is referred to herein as “physiological data.” Any one or more sensors may be used, such as a blood glucose meter, respiratory flow meter, weight scale, pulse rate monitor, or sphygmomanometer. Mobility and ease of use of the sensors are important since chronic disease patients usually take physiological measurements several times a day. Preferably the sensors are battery-powered so that they are mobile. Mobile sensors that are wearable and that communicate are known in the art, such as the Forerunner® pulse rate monitor, available from Garmin.

Each sensor 11 transmits the physiological data to a telecommunications hub 14, preferably wirelessly and preferably using the Bluetooth standard. Preferably the sensor 11 can encrypt its transmission to protect the privacy of the transmitted data.

Hub

The hub 14 receives data from the sensor 11. Preferably this communication is wireless, such as that provided by RF or infrared signals. The hub may also transmit data to the sensor which, in the typical case, will be simply a return acknowledgement signal that the physiological data was received. In addition, the hub 14 is in direct or indirect communication with a server 16, as described in more detail below. The hub 14 has memory for-internal data storage.

Preferably the hub 14 has the ability to receive and display visual and audio information sent by the server 16. For example, the hub 14 may receive customized instructions for the patient 12, educational information, customized advertisements or general advertisements that are displayed on a screen or played through a speaker. Preferably the hub 14 can also receive text messages, calendar messages and other alerts, for example Multimedia Message Service (“MMS”) messages. The receipt of these alerts is especially useful for receiving physician appointments.

Preferably the hub 14 is capable of providing a date and time stamp of the physiological data it receives from the sensor 11. This is valuable if the sensor 11 is unable to provide the date and time for a measurement.

In the preferred embodiment the hub 14 is a cell phone. As used herein “cell phone” means any phone using a cellular network, which is a radio network made up of a number of cells each served by a fixed transmitter. The hub 14 may comprise solely the receiving/transmitting device, or may optionally include web-browsing capability; a built-in camera that supports picture messaging of patient environment, eating habits, and the patient's physical condition; email capability; calendar functions; or other functions. Other devices may serve as a hub, provided they have memory and some minimal computing ability, preferably a microprocessor. Other devices that may serve as a hub include a landline telephone with a modem; a smartphone such as the Treo™; a personal digital assistant (PDA) such as the Blackberry®; hand-held, laptop, desktop personal computer; or a satellite phone. Preferably the hub 14 is a device that is easily and typically carried by the patient 12. The hub 14 may also be a wearable device, such as a bracelet worn on the wrist or upper arm. The hub 14 may also be integrated with the sensor 11.

Patients 12 may also opt to combine hubs 14 of various types to achieve communication with the sensor 11 and the server 16. For example, a patient 12 may receive physiological data on a cell phone and receive information from the server 16 on a landline. This is particularly valuable with present cell phone plans that charge patients by the minute for incoming calls: a patient obtains the wireless convenience of receiving a short message, such as the glucose measurement, on a cell phone wherever a patient is, but retains the cost-effectiveness of a landline for receiving larger messages such as medical instructions as to how to treat an elevated glucose measurement.

Patients 12 interact with the hub 14 using touch or voice recognition. Preferably the software on the hub is implemented in Java in order to run on a wide range of hubs, and uses the existing General Packet Radio Service (GPRS) network to send and receive data. Software for the hub 14 is automatically downloaded from the server 16 without patient 12 input, making implementation and updates of the system effortless to the patient 12. Methods to automatically download software to wireless devices are known in the art, such as push technologies including wireless application protocol (WAP) push service loading of a web markup language (WML) page with the installation menu, which directs the user to an over-the-air (OTA) installation method. This reduces the maintenance costs for care providers 17, since they do not have to call the patient or have physical access to the hub to perform the installation or update.

Server and Transmission Network

The hub 14 transmits data to and receives data from a central repository comprising a processing computer 20 and a database 21, referred to collectively herein as the server 16. As is known in the art, the server 16 may be a single component or an interconnected network of distributed components. Preferably the server 16 further includes an interactive voice response (IVR) 22 component.

Preferably the data is transmitted from the hub 14 to the server 16 automatically across a mobile telephone network. The preferred system uses a cellular network and transmits the data using transmission means that are supported by most cellular systems, such as the GPRS. Other transmission systems may be used, such as Circuit Switched Data (CSD) or Short Message Service (SMS).

The transmitted data is stored on the server 16. Upon receipt of the new data, the server 11 automatically analyzes the data and transmits a message to the patient 12 in response to the analysis. If the data is within preset parameters, the response may be as simple as an acknowledgement that the data was received or contain more comprehensive information. If the data deviates from preset parameters, a message is transmitted to the patient 12 with instructions customized to the deviation. A transmission sent in response to data that deviates from preset parameters is referred to herein as an “alert,” discussed in more detail below. In addition to the patient 12, a message is transmitted to a health care industry member affiliated with the patient 12. This category includes direct health care providers such as physicians, clinic staff, nurses, dieticians, clinical researchers and others authorized to provide health related recommendations to patients and indirect health care industry members including insurance companies, disease maintenance organizations and academic researchers. Collectively, the direct and indirect health care industry members affiliated with the patient 12 are referred to herein as “care providers 17.” Typically more than one care provider 17 will be in communication with the server 16 and the patient 12.

Care providers 17 set the parameters and automated responses, such as the alert levels, rules for patient reminders, doctors' appointments, and any other custom data they wish to receive or send to the patient 12. Advantages for care providers 17 include focusing efforts on patients who have poor compliance, improving patients' compliance, and spending more time with patients and less time on the phone collecting patient data. In addition, the data the care provider 17 receives is timely and accurate, resulting in a higher quality of care. The responses are transmitted to the patients 12 by text message or voice, or by personal intervention.

In the preferred embodiment, updating the server 16 with the physiological data triggers the server 16 to transmit a set of queries to the patient 12 via the hub 14 regarding the specific physiological measurement just received. For example, in response to a blood glucose reading, the system may transmit instructions to the patient 12 to navigate a graphical or voice menu on the hub 14 so that the patient 12 can input his activity level, diet, and other factors which may affect the measurement being sensed. Patients 12 may also enter subjective information about how they feel at the time of data collection. The data entered by the patient 12 is referred to herein as “patient data.”

The patient data is also recorded on the server 16 and combined with physiological data, the combination referred to herein as a “reading.” Readings are automatically analyzed by the server 16 to determine whether the reading deviates from preset parameters.

In addition to readings, the server 16 may store or have access to data related to the patient 12 such as the patients' electronic medical record and patient incentive data, explained in more detail below. Further, the systems and readings maybe integrated with patient registries or websites. For the benefit of care providers 17 responsible for multiple patients, patients can be sorted and prioritized based on this data. The data can be used in discreet portions for monitoring and responding to each patient, as well as in the aggregate for data mining. This is particularly useful for disease maintenance organizations, insurance companies, and researchers.

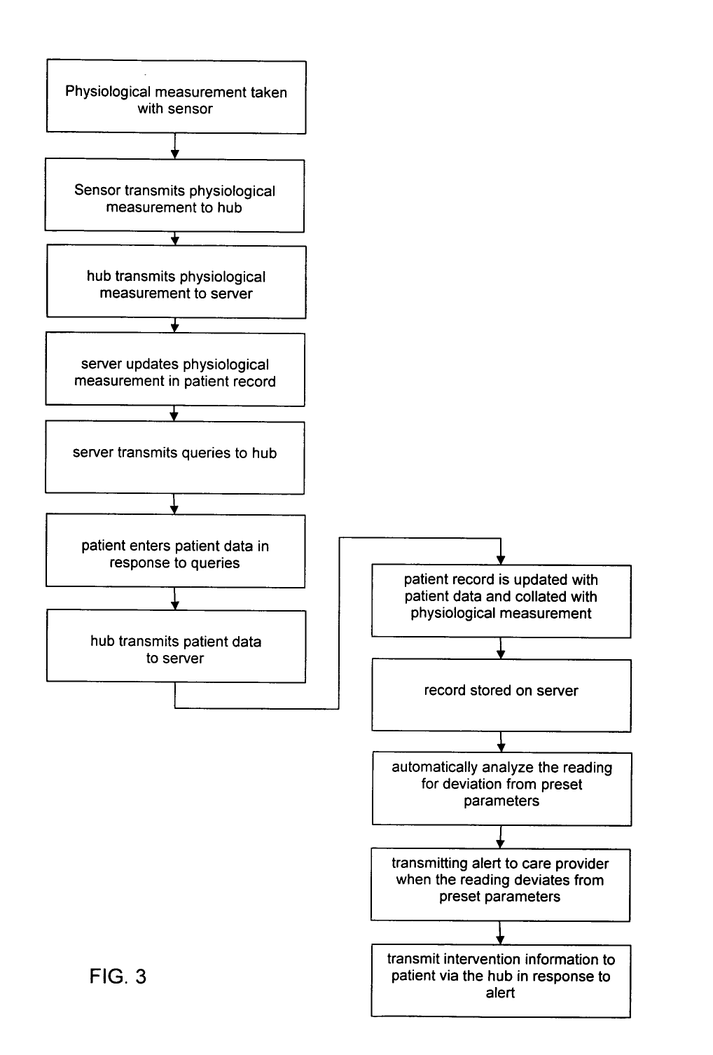

FIG. 3 illustrates a preferred implementation of the system. A physiological measurement is taken with a blood glucose sensor 11 at 3:00 in the afternoon. The sensor 11 transmits the blood glucose measurement to the hub, along with the date and time that the measurement was taken. In this case, the hub is a web-enabled cell phone with a color display. The cell phone receives the physiological data and transmits the data to the server 16. The server 16 updates the data in a patient record and automatically calls the patient on his cell phone to ask him several queries.

The patient 12 answers his cell phone and, using the IVR, the system asks the queries by voice while displaying the query on the cell phone display. The system queries, “How much have you eaten in the last two hours?” The system prompts the patient 12 for one of four answers, both by voice and visually, namely: nothing, light meal, normal meal, and heavy meal. The patient 12 responds by voice, which is recognized by the server 16, with his answer: “nothing.” Then the server 16 queries with “How much exercise have you had today?” The system prompts the patient 12 for one of four answers, namely: none, light, moderate, heavy. The patient 12 responds by voice with his answer: “light.” Finally the system queries the patient 12 both by voice and visually with “How do you feel?” and prompts with the answers: great, ok, tired, sick. The patient 12 responds by voice: “tired.”

The patient record on the server 16 is updated with this patient data and collated with the physiological measurement to form a reading, all of which is stored on the server 16. The reading is automatically analyzed by the server 16 to determine whether the reading deviates from parameters set by a disease maintenance organization (DMO) and the patient's endocrinologist. In this case, the reading deviates from preset parameters because the patient 12 should not go for two hours in the afternoon without eating. The system transmits an alert to the DMO of the outlying data and transmits intervention information automatically to the patient 12 that he needs to eat a piece of fruit immediately. At the DMO, the alert causes the DMO to review the data and, in this case, determine that while the data is deviates from the desired parameters, the patient 12 has had only one outlying reading in the past two weeks, and therefore no additional action is required.

Medication Compliance

The system is configured to help the patient take prescribed medications properly by triggering alerts when the patient is not compliant with the prescription regimen, such as the failure to take pills at the time prescribed, refills are inconsistent, or medications are not picked up. Prescription data is stored on the server 21 for each patient that indicates, at minimum, which medications are to be taken at what time. At a prescribed time, the server automatically transmits a message to the hub 14 reminding the patient to take the indicated medication. Preferably the server queries the patient about the reasons the patient is not compliant. For example, the system may ask the patient whether the cost of the medication is affordable, whether the patient is feeling undesirable side effects, or doesn't understand the directions. The server may also provide educational responses to the revealed behavior.

In addition, the server 21 may transmit a message to the hub reminding the patient to refill a medication at a given threshold. For example, assume a threshold is set at 80% of a refill quantity. If 30 pills are prescribed every month, one pill to be taken per day, the server 21 may contact the patient to suggest a refill order be placed when 24 days have passed since the last refill. The reminders may be one-time events, or are preferably transmitted until the patient takes the desired action, such as picking up a refill order. The system may further be configured to provide the patient with an opportunity to contact the pharmacy by pressing a button or by voice recognition.

Display and Incentive

The hub 14 is configured to display visual and audible information to a patient 12, such as reminders; doctors' appointments; educational information; interventional information; health information, recommendations, and goals; queries; and incentives. Preferably the hub 14 is configurable for communicating in several languages, in large text, and in color.

The hub 14 is further configured to increase compliance by displaying games that incorporate monitored conditions and providing rewards to the patient 12 when he complies with the monitoring protocol.

Care providers 17 are able to set health related goals for each patient 12 and offer incentives to achieve these goals. For example, a game on the cell phone may be used to graphically represent the patient's progress toward health related goals that are tied to incentives provided by the care providers 17. Preferably the goal is tied to a sensor 11 measurement, a more objective and verifiable value than data entered by the patient 12. Examples include losing weight, exercising regularly, and taking medication consistently. The patient 12 earns credit in the game when he takes physiological readings or correctly answers health-related questions from the server that show positive progress towards his health goals. The game is able to graphically represent the patient's progress, and once a goal is reached, notify the patient 12 of instructions on redemption of an incentive tied to that goal. The care provider 17 also receives notification generated by the system, and is able to track individual progress.

For example, a patient 12 can have a 5 pound weight loss as a goal for the month. As an incentive to reach this goal, the care provider 17 informs that patient 12 that he will receive four free music downloads when he loses the 5 pounds. The hub 14 receives and stores this goal, then translates the physiological readings generated by the patient 12 when he weighs himself into graphical representations of goal progress, via a game interface. This system validates that a goal has been met, and once met, presents the patient 12 with instructions to redeem the music downloads. Patients can also receive a health report delivered to the hub on a regular interval, indicating progress towards their goals.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiments disclosed, but that the invention will include all embodiments falling within the scope of the appended claims.

We claim:

1. A method for patient monitoring comprising:
a) sensing with a sensor at least one physiological measurement of a patient;
b) transmitting the physiological measurement wirelessly from the sensor to a telecommunications hub;
c) inputting patient data into the hub;
d) transmitting the physiological measurement and the patient data from the hub to a server;
e) storing the physiological measurement and the patient data as a reading on the server;
f) automatically analyzing the reading to determine whether the reading deviates from preset parameters;
g) transmitting an alert to one or more care providers when the reading deviates from preset parameters; and
h) transmitting intervention information to the patient via the hub in response to an alert.

2. A method for patient monitoring comprising:
a) sensing with a sensor at least one physiological measurement of a patient;
b) transmitting the physiological measurement wirelessly from the sensor to a telecommunications hub;
c) transmitting the physiological measurement from the hub to a server containing a patient record;
d) updating the patient record with the physiological measurement which triggers the server to transmit at least one query to the hub;
e) inputting patient data into the hub;
f) transmitting the patient data from the hub to the server;
g) updating the patient record with the patient data so that the sensed data and patient data are collated into a reading;
h) storing the reading on the server;
i) automatically analyzing the reading to determine when the reading deviates from preset parameters;
j) transmitting an alert to one or more care providers when the reading deviates from preset parameters; and
k) transmitting intervention information to the patient via the hub in response to an alert.

3. The method of claim 2 wherein the sensor is one of a blood glucose meter, respiratory flow meter, weight scale, pulse rate monitor, or sphygmomanometer.

4. The method of claim 2 wherein the hub is a cell phone.

5. The method of claim 2 wherein the hub comprises a microprocessor and a memory device.

6. The method of claim 2 wherein the hub comprises a land-line phone and a modem.

7. The method of claim 2 wherein inputting patient data into the hub occurs by touch or by voice.

8. The method of claim 2 further comprising displaying games on the hub for increasing compliance with a monitoring protocol.

9. The method of claim 2 further comprising transmitting a reward to the hub when a patient complies with a monitoring protocol.

10. The method of claim 2 further comprising transmitting a reminder to the hub when a patient fails to comply with a prescription regimen.

11. The method of claim 2 wherein the hub displays advertisements.

12. The method of claim 2 wherein the hub displays educational information tailored according to the patient's responses to at least one query.

13. The method of claim 2 wherein the hub further comprises a hub software application that wirelessly receives updates from the server.

14. The method of claim 2 wherein the care provider is a disease maintenance organization.

15. The method of claim 2 wherein the care provider is an insurance company.

16. The method of claim 2 wherein the care provider is a health care provider.

17. The method of claim 2 wherein the hub is worn by the patient.

18. The method of claim 2 wherein the hub is a bracelet.

19. A patient monitoring system comprising a sensor, a wireless telecommunications hub, and a server wherein:
a) the sensor transmits sensed data wirelessly to the hub;
b) the hub:
i. receives the sensed data from the sensor;
ii. transmits the sensed data to the server;
iii. receives visual and audio information tailored to the patient from the server, wherein the information further comprises at least one query;
iv. receives patient data that is input by the patient in response to the queries;
v. transmits the patient data to the server; and
c) the server:
i. collates the sensed data and patient data into a reading;
ii. stores the reading;
iii. automatically analyzes the reading to determine when the reading deviates from preset parameters;
iv. transmits an alert to one or more care providers when the reading deviates from preset parameters; and
v. transmits information to the patient via the hub in response to an alert.

20. The system of claim 19 wherein the hub displays games for increasing compliance with a monitoring protocol.

21. The system of claim 19 wherein the hub transmits rewards to a patient for complying with a monitoring protocol.

22. The system of claim 19 further comprising transmitting a reminder to the hub when a patient fails to comply with a prescription regimen.

23. The system of claim 19 wherein the hub displays advertisements.

24. The system of claim 19 wherein the hub displays educational information tailored according to the patient's responses to at least one query.

25. The system of claim 19 wherein the hub further comprises a hub software application that wirelessly receives updates from the server.

26. The system of claim 19 wherein the care provider is a disease maintenance organization.

27. The system of claim 19 wherein the care provider is an insurance company.

28. The system of claim 19 wherein the care provider is a health care provider.

29. The system of claim 19 wherein the hub is worn by the patient.

30. The system of claim 19 wherein the hub is a bracelet.

31. The system of claim 19 wherein the hub is integrated with the sensor.
